(19) United States
(12) Patent Application Publication
(10) Pub. No.: US 2022/0151902 A1
(43) Pub. Date: May 19, 2022

(54) ENCAPSULATED COMPOSITION

(71) Applicant: GIVAUDAN SA, Vernier (CH)

(72) Inventor: Marion DENIGOT, Maisons Laffitte (FR)

(21) Appl. No.: 17/440,449

(22) PCT Filed: Mar. 31, 2020
(86) PCT No.: PCT/EP2020/059028

§ 371 Date: Sep. 17, 2021

(30) Foreign Application Priority Data
Apr. 8, 2019 (GB) 1904918.8

(51) Int. Cl. A61K 8/58; A61K 8/11; A61Q 5/12; A61Q 5/02; A61Q 19/10; A61Q 19/00; A61K 8/81; B01J 13/16; B01J 13/22; C11D 3/50

(52) U.S. Cl. CPC A61K 8/585; A61K 8/11; A61Q 5/12; A61Q 5/02; A61Q 19/10; A61K 2800/52; A61K 8/8164; B01J 13/16; B01J 13/22; C11D 3/505; A61K 2800/56; A61Q 19/00

(57) ABSTRACT

Described is an encapsulated composition comprising at least one core-shell microcapsule. The at least one core-shell microcapsule comprises a core comprising at least one perfume and/or cosmetic ingredient, and a shell surrounding the core. The shell comprises a polymeric stabilizer that is formed by combination of a polymeric surfactant with at least one bipodal aminosilane. Disclosed is also a method of preparing an encapsulated composition and a use of such an encapsulated composition to enhance the performance of perfume and/or cosmetic ingredients in consumer goods.

The present invention is concerned with an encapsulated composition comprising at least one core-shell microcapsule. The invention also relates to a method of preparing an encapsulated composition and to a use of such a composition to enhance the performance of perfume and/or cosmetic ingredients in consumer goods. Furthermore, the present invention refers to a polymeric stabilizer, as well as to a use of such a polymeric stabilizer in the encapsulation of perfume and/or cosmetic ingredients.

It is known to incorporate encapsulated functional materials in consumer products, such as household care, personal care, and fabric care products. Functional materials include for example fragrances, cosmetic agents, drugs and substrate enhancers.

Microcapsules that are particularly suitable for delivery of such functional materials are core-shell microcapsules, wherein the core usually comprises the functional material and the shell is impervious or partially impervious to the functional material. Generally, these microcapsules are used in aqueous media and the encapsulated ingredients are hydrophobic. A broad selection of shell materials can be used, provided the shell material is impervious or partially impervious to the encapsulated ingredient.

Among the functional materials, perfume and/or cosmetic ingredients are encapsulated for a variety of reasons. Microcapsules can isolate and protect this kind of material from external suspending media, such as consumer product bases, with which they may be incompatible or unstable in. They are also used to assist in the deposition of the ingredients onto substrates, such as skin or hair, or also fabrics or hard household surfaces in case of perfume ingredients. They can also act as a means of controlling the spatio-temporal release of an ingredient.

A wide variety of encapsulating media as well as perfume and/or cosmetic ingredients suitable for the preparation of encapsulated compositions has been proposed in the prior art. Such encapsulating media include synthetic resins made from polyamides, polyureas, polyurethanes, polyacrylates, melamine-derived resins, or mixtures thereof.

As for suitable core materials, in principal, all ingredients on the perfume and/or cosmetics palette can be incorporated to some extent into a core-shell microcapsule. However, it is generally accepted that certain physico-chemical characteristics of an ingredient, most notably its clog P, will influence whether and to what extent it can be encapsulated, and once encapsulated, its propensity to remain in the core without substantial leakage during storage.

In the hands of the skilled formulator, the judicious selection of both the shell and core materials can result in microencapsulated compositions that are stable in many consumer products, which allows modulating the release of fragrance and/or cosmetics over time. However, even the use of well-established shell chemistries in combination with an appropriate formulation in the core, the formulator is faced with a difficult trade-off between ensuring on one hand that the microcapsules are sufficiently robust as to be stable and not leaky during manufacture and storage, and on the other that there is sufficient release of the core contents in application. Another problematic aspect is undesired side reactions of shell-forming compounds with the materials to be encapsulated during capsule formation.

US 2014/0331414 A1 discloses microcapsules obtained by emulsifying a perfume oil in the presence of a polymeric surfactant and silanes. The advantage of this chemistry is that it proceeds under mild reaction conditions, in particular that the silanes are relatively unreactive towards the substances to be encapsulated. These resulting microcapsules show good olfactive performance on wet fabrics, but exhibit limited stability with respect to leakage of the perfume ingredients in product bases comprising surfactants under prolonged storage conditions.

It is therefore a problem underlying the present invention to overcome the above-mentioned shortcomings in the prior art. In particular, it is a problem underlying the present invention to provide encapsulated compositions of the above-mentioned kind that show increased stability during manufacture and storage, but keep a desired release profile during application. Furthermore, the compositions should be producible in an operationally safe, robust and cost-efficient process.

These problems are solved by the present invention. In a first aspect, this invention relates to an encapsulated composition comprising at least one core-shell microcapsule. The at least one core-shell microcapsule comprises a core comprising at least one perfume and/or cosmetic ingredient, and a shell surrounding the core. The shell comprises a polymeric stabilizer that is formed by combination of a polymeric surfactant with at least one bipodal aminosilane.

Under “polymeric surfactant” it is understood a polymer that has the ability to lower the interfacial tension between an oil phase and an aqueous phase, when dissolved in one or both of the phases. This ability to lower interfacial tension is called “interfacial activity”.

By “bipodal aminosilane” is meant a molecule comprising at least one amino group and two residues, each of these residues bearing at least one alkoxysilane moiety.

Under “formed by combination” it is understood in the present context that the polymeric surfactant and the at least one bipodal aminosilane are brought in contact with each other to generate the polymeric stabilizer. Without being bound to any theory, this formation can be the result of an interaction between the polymeric surfactant and the at least one bipodal aminosilane, such as dispersion forces, electrostatic forces or hydrogen bonds. But also a chemical reaction to form covalent bonds is encompassed by this term.

In addressing the problems of the prior art, it has been discovered that the polymeric stabilizer is a contributing factor to the balance between microcapsule stability with respect to perfume leakage during storage and perfume release under in-use conditions. In particular, the importance of providing additional stabilization of the oil-water interface has been recognized.

The polymeric stabilizer helps to provide a particularly stable platform on which to deposit various shell chemistries around perfume oil droplets to form novel encapsulated perfume compositions, which provide the formulator with greater latitude to design microcapsules with additional functionality or desirable properties.

In particular embodiments of the present invention, the at least one bipodal aminosilane has the Formula (I).

(O—R⁴)_{(3-f)}(R³)_{f}Si—R²—X—R²—Si(O—R⁴)_{(3-f)}(R³)_{f} Formula (I)

In the above Formula (I), X stands for —NR¹—, —NR¹—CH₂—NR¹—, —NR¹—CH₂—CH₂—NR¹—, —NR¹—CO—NR¹—, or

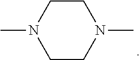

In the above Formula (I), R¹ each independently stands for H, CH₃ or C₂H₅. R² each independently stands for a linear or branched alkylene group with 1 to 6 carbon atoms. R³ each independently stands for a linear or branched alkyl group with 1 to 4 carbon atoms. R⁴ each independently stands for H or for a linear or branched alkyl group with 1 to 4 carbon atoms. f stands for 0, 1 or 2.

The reason why bipodal aminosilanes are particularly advantageous for forming stable oil-water interfaces, compared to conventional silanes, is not fully understood. Without being bound by any theory, it may be suggested that this beneficial role is linked to the particular, bi-directional arrangement of the silane moieties in the molecule.

Typical examples of bipodal aminosilanes include bis(3-(triethoxysilyl)propyl)amine, N,N′-bis(3-(trimethoxysilyl)propyl)urea, bis(3-(methyldiethoxysilyl)propyl)amine, N,N′-bis(3-(trimethoxysilyl)propyl)ethane-1,2-diamine, bis(3-(methyldimethoxysilyl)propyl)-N-methylamine and N,N′-bis(3-(triethoxysilyl)propyl)piperazine.

In preferred embodiments of the present invention, the bipodal aminosilane is a secondary aminosilane. Using a secondary bipodal aminosilane instead of primary aminosilane decreases the reactivity of the stabilizer with respect to electrophilic species, in particular aldehydes. Hence, perfumes containing high levels of aldehydes may be encapsulated easily.

In particular embodiments of the present invention, the bipodal secondary aminosilane is bis(3-(triethoxysilyl)propyl)amine. This particular secondary aminosilane has the advantage of releasing ethanol instead of more toxic and less desirable methanol during the mutual polycondensation of the ethoxysilane groups.

In particular embodiments of the present invention, the polymeric stabilizer is formed by combination of the polymeric surfactant with the at least one bipodal aminosilane and a further aminosilane, preferably an aromatic aminosilane, even more preferably selected from the group consisting of compounds having Formula (II).

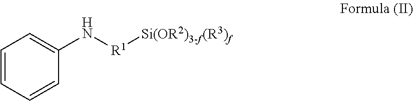

In the above Formula (II), R¹ stands for a linear or branched alkylene group with 1 to 6 carbon atoms. R² each independently stands for a linear or branched alkyl group with 1 to 4 carbon atoms. R³ each independently stands for H or for a linear or branched alkyl group with 1 to 4 carbon atoms. f stands for 0, 1, or 2.

It has been found that including a further aminosilane in the polymeric stabilizer results in a particularly stable water-oil interface.

In particular embodiments of the present invention, the aromatic aminosilane is selected from the group consisting of N-(3-(trimethoxysilyl)propyl)aniline and N-((trimethoxysilyl)methy)aniline.

In particular embodiments of the present invention, the polymeric stabilizer is formed by combination of the polymeric surfactant with the at least one bipodal aminosilane and a tripodal aminosilane. The tripodal aminosilane can be an aminosilane of Formula (III).

N[R²Si(OR⁴)_{(3-f)}(R³)_{f}]₃ Formula (III)

In the above Formula (III), R² each independently stands for a linear or branched alkylene group with 1 to 6 carbon atoms. R³ each independently stands for a linear or branched alkyl group with 1 to 4 carbon atoms. R⁴ each independently stands for an H or for a linear or branched alkyl group with 1 to 4 carbon atoms. f stands for 0, 1 or 2.

In particular embodiments of the present invention, the polymeric stabilizer is formed by combination of the polymeric surfactant with the at least one bipodal aminosilane and a tripodal aminosilane of Formula (IV).

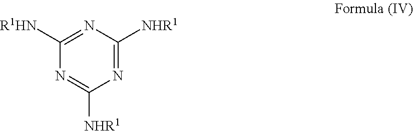

In the above Formula (IV), R¹ stands for R²Si(O—R⁴)_{(3-f)}(R³)_{f}. R² each independently stands for a linear or branched alkylene group with 1 to 6 carbon atoms. R³ each independently stands for a linear or branched alkyl group with 1 to 4 carbon atoms. R⁴ each independently stands for an H or for a linear or branched alkyl group with 1 to 4 carbon atoms. f stands for 0, 1 or 2.

Tripodal aminosilanes have the advantage of providing additional cross-linking possibilities within the shell.

In the context of the present invention, the polymeric surfactant is preferably soluble in the aqueous phase. Surfactants soluble in the aqueous phase favor the formation of oil-in-water emulsions.

A convenient way to assess the interfacial activity of a polymeric surfactant that is soluble in an aqueous phase is to measure the tension of the interface between the aqueous phase comprising the polymeric surfactant and air. This tension is called “surface tension” and is generally expressed in mN/m. The surface tension may be measured by a number of methods which are well known to the skilled person. For example, the surface tension may be measured by measuring the force necessary to separate a platinum ring of known circumference from the surface of the aqueous phase, using a so-called Du Noüy ring tensiometer. Alternatively, the surface tension may be obtained from the force required to wet a platinum or glass plate oriented perpendicularly to the surface of the aqueous phase, according to the so-called Wilhelmy plate method.

For a given polymeric surfactant, the surface tension depends on the temperature and on the concentration of this polymeric surfactant in the aqueous phase. Furthermore, if the polymeric surfactant is a polyelectrolyte comprising cationic groups or anionic groups or a polymer comprising groups that can form cations or anions, the surface tension additionally depends on the ionic strength and/or on the pH of the aqueous phase. The surface tension of pure water is about 72 mN/m at 25° C.

The surface tension of an aqueous phase comprising a polymeric surfactant may also depend on the age of the surface, due to slow molecular motions and rearrangements at the interface.

In particular embodiments of the present invention, the polymeric surfactant is a polymer causing a surface tension of lower than 60 mN/m, more particularly lower than 55 mN/m, still more particularly lower than 50 mN/m, in a 1 wt.-% aqueous solution containing 0.01 wt.-% of sodium chloride, when measured after 1 hour of equilibration at at least one pH value selected from 3.5, 4.0, 4.5, 5.0, 5.5, 6.0 and 6.5 at a temperature of 25° C. with a Du Noüy ring tensiometer (Type: K100 from Krüss GmbH, Germany). The concentrations are in weight percent of the total weight of aqueous phase.

Interfacial activity is expected to drive the polymer toward the oil/water interface where the polymeric stabilizer is formed.

Preferably, the polymeric surfactant comprises anionic groups or groups that can form anions, such as sulfate groups, sulfonate groups, phosphate groups, carboxylic acid groups and anhydride groups. Without being bound by any theory, it is surmised that negatively charged polymers may interact favorably with the aminosilanes mentioned hereinabove.

In particular embodiments of the present invention, the polymeric surfactant is a co-polymer of maleic anhydride and ethylene and/or vinyl methyl ether. Such copolymers have high interfacial activity owing to the coexistence of both hydrophilic and hydrophobic moieties.

The co-polymer of maleic anhydride and ethylene and/or vinyl methyl ether can be alternate. Alternate co-polymers are preferred over block and random copolymers because of the more homogenous distribution of the maleic moieties along the main polymer chain.

In particular embodiments of the present invention, the co-polymer of maleic anhydride and ethylene and/or vinyl methyl ether is fully or partially hydrolyzed.

Without being bound by any theory, the applicant believes that the silane groups further polycondensate with one another to form a silica network at the interface that additionally stabilizes this interface.

In particular embodiments of the present invention, the polymeric stabilizer is formed by combination of bis(3-(triethoxysilyl)propyl)amine with (3-(trimethoxysilyl)propyl)aniline and fully or partially hydrolyzed poly(ethylene-maleic anhydride) and/or poly (vinylmethylether-co-maleic anhydride), in particular in the alternate form. This combination of bipodal secondary aminosilane and aromatic secondary aminosilane provides the desired interface stability and barrier properties. The stabilized interface becomes sufficiently impervious to effectively encapsulate the at least one perfume ingredient comprised in the core. The polymeric stabilizer effectively forms a shell encapsulating the at least one perfume ingredient comprised in the core.

In encapsulated compositions according to the present invention, the bipodal aminosilane to polymeric surfactant weight ratio can be from 0.02 to 1, in particular from 0.2 to 0.9, even more particularly from 0.3 to 0.7. The further aminosilane to polymeric surfactant weight ratio can be from 0.02 to 1, in particular from 0.1 to 0.7, even more particularly from 0.15 to 0.5. The shell to core weight ratio can be from 0.015 to 0.2, in particular from 0.03 to 0.09, even more particularly from 0.04 to 0.07.

Microcapsules mentioned hereinabove may be used as such or in combination with additional shell-forming materials to form a second shell encapsulating the first shell mentioned hereinabove, which thereby becomes a first shell underlying the second shell.

In particular embodiments of the present invention, the additional shell comprises at least one shell-forming material obtainable by:
Reacting of an alkylolated polyfunctional amine or reacting of a polyfunctional amine with an aldehyde;
Reacting a polyisocyanate and a polyfunctional amine;
Reacting a polyfunctional amine and a polyfunctional (meth)acrylate; and/or
Reacting unsaturated monomers selected from the group consisting of styrene, divinylbenzene, alkyl (meth)acrilyates, polyfunctional (meth)acrylates, and vinyl monomes.

A second aspect of the present invention relates to a method of preparing an encapsulated composition, in particular a composition as described herein above. The method comprises the steps of:
a. Dissolving a polymeric surfactant in an aqueous phase;
b. Dissolving at least one bipodal aminosilane in an oil phase comprising at least one perfume and/or cosmetic ingredient;
c. Emulsifying the oil phase into the aqueous phase to form an oil-in-water emulsion;
d. Causing the bipodal aminosilane and the polymeric surfactant to form a first shell of polymeric stabilizer encapsulating the dispersed oil droplets, thereby forming a slurry of microcapsules.

Oil-in-water emulsions have the advantage of providing a plurality of droplets that may be used as template for shell formation, wherein the shell is built around each of these droplets. Additionally, the droplet size distribution may be controlled in emulsions, by controlling the conditions of emulsifications, such as stirring speed and stirrer geometry. As a result, a plurality of microcapsules is obtained with controlled average size and size distribution, wherein the oil phase is encapsulated and forms thereby the core of the microcapsules.

With respect to step d., the formation of the first shell of polymeric stabilizer is preferably initiated by adjusting the pH to a range of from 4.0 to 5.0. The temperature is preferably maintained at room temperature for at least 1 h, more preferably at least 2 h, even more preferably at least 3 h, for example 3.5 h, and then increased to at least 60° C., preferably at least 70° C., more preferably at least 80° C., but not more than 90° C. Under these conditions, the formation of the shell is well controlled, meaning optimal stabilization of the interface is obtained.

The appropriate stirring speed and geometry of the mixer can be selected in order to obtain the desired average droplet size and droplet size distribution. It is a characteristic of the present invention that the polymeric stabilizer has sufficient surfactant power and is able to promote the formation of dispersed oil droplets with desirable small droplet size and low polydispersity.

In a process according to the present invention, a one-liter vessel equipped with a turbine, or a cross-beam stirrer with pitched beam, such as a Mig stirrer, and having a stirrer diameter to reactor diameter of 0.6 to 0.8 may be used. Microcapsules can be formed in such reactor having an average particle size D(50) of 30 microns or less, more particularly 20 microns or less, and with a polydispersity span of less than 1.5, more particularly less than 1.3, still more particularly less than 1.2, at a stirring speed of less than 1000 rpm, more particularly in the order of from about 100 to about 1000 rpm, still more particularly from about 500 to 700 rpm, for example 600 rpm. Preferably, a Mig stirrer is used operating at a speed of 600±50 rpm. The person skilled in the art will however easily understand that such stirring conditions may change depending on the size of the reactor and of the volume of the slurry, on the exact geometry of the stirrer on the ratio of the diameter of the stirrer to the diameter of the reactor diameter ratios. For example, for a Mig stirrer with stirrer to reactor diameter ratio from 0.5 to 0.9 and slurry volumes ranging from 0.5 to 8 tons, the preferable agitation speed in the context of the present invention is from 150 rpm to 50 rpm.

In a particular embodiment of the present invention, the bipodal aminosilane to polymeric surfactant weight ratio in the emulsion is set within a range of from 0.02 to 1, more particularly from 0.2 to 0.9, still more particularly from 0.3 to 0.7, for example 0.35 or 0.65.

In a particular embodiment of the present invention, a further aminosilane, preferably an aromatic aminosilane, is additionally dissolved in the oil phase (above-described step b.) to form a first shell of polymeric stabilizer encapsulating the dispersed oil droplets (above-described step d.).

The further aminosilane to polymeric surfactant weight ratio in the emulsion can be set within a range of from 0.2 to 0.7, in particular particularly from 0.3 to 0.5, for example 0.35.

In a particular embodiment of the present invention, the shell material to oil ratio in the emulsion is set within a range from 0.015 to 0.2, more particularly from 0.03 to 0.09, still more particularly from 0.04 to 0.07, for example 0.06.

Microcapsules obtainable by the process mentioned hereinabove may be used as such or serve as a first shell on which a second shell comprising at least one additional shell-forming material may be formed.

The additional shell-forming materials may be added following the formation of the aforementioned shell of polymeric stabilizer. The process may then comprise the steps of:
a. Dissolving a polymeric surfactant in an aqueous phase;
b. Dissolving at least one bipodal aminosilane in an oil phase comprising at least one perfume and/or cosmetic ingredient;
c. Emulsifying the oil phase into the aqueous phase to form an oil-in-water emulsion;
d. Causing the bipodal aminosilane and the polymeric surfactant to form a shell of polymeric stabilizer encapsulating the dispersed oil droplets, forming thereby a slurry of microcapsules;
e. Providing additional shell-forming materials and causing these additional shell-forming materials to react and to form an additional shell encapsulating the microcapsules formed in step d.

After formation of the microcapsules, the encapsulated composition is usually cooled to room temperature. Before, during or after cooling, the encapsulated composition may be further processed. Further processing may include treatment of the composition with anti-microbial preservatives, which preservatives are well known in the art. Further processing may also include the addition of a suspending aid, such as a hydrocolloid suspending aid to assist in the stable physical dispersion of the microcapsules and prevent any creaming or coalescence. Any additional adjuvants conventional in the art may also be added at this time.

The case where a polyfunctional amine and a polyisocyanate are used as additional shell-forming monomers constitutes a particular process of forming an encapsulated composition of the present invention. The process can comprise the steps of:
a. Forming a slurry of microcapsules having a first shell comprising the polymeric stabilizer according to the present invention, as mentioned herein above;
b. Adding at least one polyisocyanate, in particular adding a polyisocyanate (A) and a polyisocyanate (B), which is different from polyisocyanate (A);
c. Adding at least one polyfunctional amine;
d. Effecting formation a second shell around first shell formed in step a.

In the above process, the pH of the aqueous phase of the slurry formed in step a. can be adjusted to a range of from 4 to 8, preferably from 5 to 7, for example around 6. The pH can be adjusted using an inorganic base, for example sodium hydroxide solution, or carbonate buffer salts.

Organic isocyanates are compounds in which an isocyanate group is bonded to an organic residue (R—N═C═O or R—NCO). In the context of the present invention, polyisocyanates (or polyfunctional isocyanates) are organic isocyanates with two or more (e.g. 3, 4, 5, etc.) isocyanate groups in a molecule. Suitable polyisocyanates are, for instance, aromatic, alicyclic or aliphatic.

Polyisocynate A mentioned herein above is preferably an anionically modified polyisocyanate, which comprises at least two isocyanate groups and at least one functional group which is anionic or anionogenic. An “anionogenic functional group” is a group which can become anionic depending on the chemical environment, for instance the pH. Suitable anionic or anionogenic groups are, for instance, carboxylic acid groups, sulfonic acid groups, phosphonic acid groups and salts thereof. Suitable salts can be sodium, potassium or ammonium salts. Ammonium salts are preferred.

Anionically modified polyisocyanate A can be selected in each case from anionically modified hexamethylene diisocyanate, isophorone diisocyanate, dicyclohexylmethane-4,4′-diisocyanate, the isocyanurate of hexamethylene diisocyanate and mixtures thereof.

In a particularly preferred embodiment, the anionically modified polyisocyanate A is a modified isocyanurate of hexamethylene diisocyanate, sold by Covestro under the trademark Bayhydur® XP2547.

In one aspect of the present invention, polyisocyanate B can be a non-ionic polyisocyanate.

Preferably, non-ionic polyisocyanate B is selected from the group consisting of hexamethylene diisocyanate, tetramethylene diisocyanate, dicyclohexylmethane-4,4′-diisocyanate, 2,4- and 2,6-toluylene diisocyanate and isomer mixtures thereof, 2,4′- and 4,4′-diphenylmethane diisocyanate and isomer mixtures thereof, xylylene diisocyanate (for example Desmodur® quix 175 sold by Covestro), optionally as a trimethylolpropane (TMP) adduct (for example commercially available under the trademark Takenate™ D-110N), the biurets, allophanates and/or isocyanurates of the afore-mentioned polyisocyanates or mixtures thereof.

A preferred commercially available non-ionic polyisocyanate B is dicyclohexylmethane diisocyanate, in particular sold by Covestro AG under the trademark Desmodur® W1.

A preferred commercially available non-ionic polyisocyanate B is hexamethylene diisocyanate, in particular sold by Covestro AG under the trademark Desmodur® N3200.

A preferred commercially available non-ionic polyisocyanate B is isophorone diisocyanate, in particular sold by Covestro AG under the trademark Desmodur® Z.

These polyisocyanates have the advantage of being non-aromatic and therefore more sustainable and less prone to oxidation, while still having high reactivity with polyamines and suitable molecular structure for the formation of impervious encapsulating resins.

The weight ratio of anionically modified polyisocyanate A to non-ionic polyisocyanate B can be in the range from 10:1 to 1:10, more preferably in the range from 1:1 to 1:5 and in particular in the range from 1:2 to 1:4. These weight ratios provide resins having the highest imperviousness and therefore the most suitable for encapsulation.

In the present context, the term “polyfunctional amine” denotes amines that comprise at least two groups capable of reacting with NCO groups, wherein at least one of the groups capable of reacting with NCO groups is a primary or secondary amino group.

The polyfunctional amine is preferably selected from diamines, triamines, tetramines, and higher order polyfunctional amines, aminoalcohols, melamines, urea, hydrazines, polymeric polyamines, and mixtures thereof.

Preference is given to polymeric polyamines having a weight-average molecular weight of at least 300 g/mol. More preferred are polymeric polyamines having a weight-average molecular weight of from 500 to 2 000 000 g/mol, in particular from 700 to 1 000 000 g/mol, even more particularly from 800 to 500 000 g/mol.

Preferred commercially available polyethylenimines are sold by BASF SE under the trademark Lupasol®, particularly Lupasol™ G100.

It is preferred to use polyethyleneimine and isocyanate compounds in a weight ratio of 1:1 to 1:5, especially 1:2 to 1:3, or in a dry weight ratio of 1:1 to 1:10, especially 1:4 to 1:6. These weight ratios provide resins having the highest encapsulation efficiency and therefore the most suitable for encapsulation.

Formation of the shells around the droplets in step d. can be effected by heating. This can be achieved at a temperature of at least 50° C., preferably at least 60° C., more preferably in a range of from 65° C. to 90° C., in order to ensure sufficiently rapid reaction progress. It may be preferred to increase the temperature continuously or in stages (e.g. in each case by 5° C.) until the reaction is essentially complete. Afterwards, the dispersion may cool down to room temperature.

The reaction time typically depends on the nature of the reactive wall-forming materials, the amount of said materials employed, and the temperature used. The period of time for the reaction is ranging from a few minutes to several hours. Usually, microcapsule formation is effected between ca. 60 minutes to 6 h or up to 8 h at the temperatures defined above.

In accordance with another particular embodiment of the present invention, the additional shell-forming monomers may be selected from polyfunctional amine pre-condensates, more particularly melamine and urea pre-condensates with aldehydes, and particularly formaldehyde.

A process for preparing a respective encapsulated composition may comprise the steps of:
a. Forming a slurry of microcapsules having a first shell comprising the polymeric stabilizer according to the present invention, as mentioned herein above;
b. Adding at least one polyfunctional amine pre-condensate;
c. Effecting formation a second shell around first shell formed in step a.

The pH range of the reaction in step c. is in the acidic domain, more particularly between 3 and 6, for example 4.4±0.5 and the reaction temperature is from about 50° C. to 95° C., more particularly from 70° C. to 90° C. Additionally, a formaldehyde scavenger may be employed to reduce the level of formaldehyde in the final slurry, wherein the formaldehyde scavenger may be added before, during or after the slurry is cooled down to room temperature.

In accordance with the process of the present invention, if desired, a functional coating can be applied to the first or to the second shell of the core-shell microcapsules. A functional coating may entirely or only partially coat the microcapsule shell. Whether the functional coating is charged or uncharged, its primary purpose is to alter the surface properties of the microcapsule to achieve a desirable effect, such as to enhance the deposition of the microcapsule on a treated surface, such as a fabric, human skin or hair. Functional coatings may be post-coated to already formed microcapsules, or they may be physically incorporated into the microcapsule shell during shell formation. They may be attached to the shell by physical forces, physical interactions, such as hydrogen bonding, ionic interactions, hydrophobic interactions, electron transfer interactions, or they may be covalently bonded to the shell.

If the functional coating is to be attached to the shell by physical association, the chemical structure of the coating will to some extent be determined by its compatibility with the shell chemistry, since there has to be some association to the microcapsule shell. If the functional coating is to be covalently bound to the shell, this may be facilitated by incorporating into the shell, materials bearing functional groups that are able to react with the coating material.

Suitable coating materials may be based on polysaccharides, polypeptides, polycarbonates, polyesters, polyolefinic (vinyl, acrylic, acrylamide, polydiene), polyester, polyether, polyurethane, polyoxazoline, polyamine, silicone, polyphosphazine, polyaromatic, polyheterocyclic. A more detailed list of coating materials can be found in the patent literature, for example EP 1 797 947 A2, which discloses coating materials that can be employed as deposition aids.

Particularly preferred coating materials may be selected from the group consisting of polymethyl(meth)acrylate, polydimethylaminoethyl(meth)acrylate, polybutyl(meth)acrylate, polydiallydimethylammonium chloride, and mixtures thereof.

If the coating material is a polymer, it can be generated in-situ during the coating process by the polymerization of coating material monomers. More particularly, suitable monomers can be added to a slurry of core-shell microcapsules formed according to a process described herein and caused to polymerize as well as to react with functional groups on the shell, if applicable, in order to build-up polymeric coating material that is covalently bound to the shell, and which at least partially coats it.

Accordingly, in a particular embodiment of the present invention, there is provided a method of forming a microcapsule and an encapsulated composition containing same, said method comprising the steps of:
a. Forming a microcapsule slurry in accordance with any of the processes described hereinabove;
b. Adding a polymerizable monomer to the slurry and causing the monomer to both polymerize and react with functional groups available on the microcapsule shells to form a coating material covalently bound to the shells of the core-shell microcapsules.

The coating polymer can be a cationic or an cationic ampholytic polymer. In the context of the present invention, an “ampholytic polymer” is to be understood as a polymer comprising both cationic and anionic groups, or comprising corresponding ionizable groups. A cationic ampholytic polymer comprises more cationic groups than anionic groups or groups that can form anions, and as such, has a net positive charge.

The ampholytic polymer can comprise from 1 to 99 mol % of cationic groups and from 1 to 99 mol % of anionic groups or groups than can form an anion. In a preferred embodiment of the present invention, the ampholytic polymer comprises 2 to 99 mol %, in particular 30 to 95 mol %, and more particularly 60 to 90 mol %, of cationic groups and 1 to 98 mol %, in particular 5 to 70 mol %, and more particularly 10 to 40 mol % of anionic groups or groups than can form an anion.

The cationic groups in the cationic polymer can be pH independent. The cationic groups in the cationic polymer can be quaternary ammonium groups.

The cationic polymer can be derived from at least one a monomer bearing quaternary ammonium functionality. In particular, the cationic monomer can be selected from the group consisting of quaternized dimethylaminoethyl acrylate (ADAME), quaternized dimethylaminoethyl methacrylate (MADAME), dimethyldiallylammonium chloride (DADMAC), acrylamidopropyltrimethylammonium chloride (APTAC) and methacrylamidopropyltrimethylammonium chloride (MAPTAC).

When the cationic polymer comprises anionic groups or groups that can form anions, it can be additionally derived from a monomer selected from the group consisting of acrylic based monomers, including acrylic acid, methacrylic acid, itaconic acid, crotonic acid, maleic acid, fumaric acid and strong-acid monomers, for example monomers with a sulfonic or a phosphonic acid-type function such as 2-acrylamido-2-methylpropane sulfonic acid, vinylsulfonic acid, vinylphosphonic acid, allylsulfonic acid, allylphosphonic acid, styrene sulfonic acid. The acrylic based monomer may also be any water-soluble salts of these monomers wherein the salt is a salt of an alkali metal, an alkaline-earth metal or an ammonium. The most preferred acrylic based monomer is acrylic acid, methacrylic acid, or a water soluble salt thereof.

The cationic polymer can further be additionally derived from a non-ionic monomer selected from the group consisting of water soluble vinyl monomers, more particularly acrylamide, methacrylamide, N-isopropylacrylamide, N,N-dimethylacrylamide, N-methylolacrylamide, N-vinylformamide, N-vinyl acetamide, N-vinylpyridine and/or N-vinylpyrrolidone.

The cationic polymer can be an ampholytic co-polymer derived from a cationic monomer or a monomer that can form cations, in particular containing at least one quaternary ammonium group, an anionic monomer or a monomer that can form anions, in particular based on acrylic acid, methacrylic acid or a derivative thereof, and optionally a non-ionic monomer. Such polymers offer an optimal combination of being compatible with the shell, having good dispersion efficiency, good flow properties and excellent affinity with the various substrates hereinabove mentioned.

In a more particular embodiment, the ampholytic co-polymer is a co-polymer of acrylic acid dimethyldiallyl ammonium chloride (DADMAC).

The ampholytic polymer may be employed in an encapsulated composition according to the present invention in an amount from 1 to 20 wt.-%, more particularly 2 to 10 wt.-%, based on the weight of the composition.

The ampholytic polymer can be prepared using polymerization techniques that are well known to a person skilled in the art. These known polymerization techniques include solution polymerization, gel polymerization, precipitation polymerization, inverse emulsion polymerization, aqueous emulsion polymerization, suspension polymerization and micellar polymerization.

In particular embodiments of the present invention, the at least one perfume ingredient is selected from the group consisting of ADOXAL™ (2,6,10-trimethylundec-9-enal); AGRUMEX™ (2-(tert-butyl)cyclohexyl acetate); ALDEHYDE C 10 DECYLIC (decanal); ALDEHYDE C 11 MOA (2-methyldecanal); ALDEHYDE C 11 UNDECYLENIC (undec-10-enal); ALDEHYDE C 110 UNDECYLIC (undecanal); ALDEHYDE C 12 LAURIC (dodecanal); ALDEHYDE C 12 MNA PURE (2-methylundecanal); ALDEHYDE ISO C 11 ((E)-undec-9-enal); ALDEHYDE MANDARINE 10%/TEC ((E)-dodec-2-enal); ALLYL AMYL GLYCOLATE (allyl 2-(isopentyloxy)acetate); ALLYL CYCLOHEXYL PROPIONATE (allyl 3-cyclohexylpropanoate); ALLYL OENANTHATE (allyl heptanoate); AMBER CORE™ (1-((2-(tert-butyl)cyclohexyl)oxy)butan-2-ol); AMBERMAX™ (1,3,4,5,6,7-hexahydro-beta,1,1,5,5-pentamethyl-2H-2,4a-methanonaphthal-ene-8-ethanol); AMYL SALICYLATE (pentyl 2-hydroxybenzoate); APHERMATE (1-(3,3-dimethylcyclohexyl)ethyl formate); BELAMBRE™ ((1R,2S,4R)-2′-isopropyl-1,7,7-trimethylspiro[bicyclo[2.2.1]heptane-2,4′-[1,3]dioxane]); BIGARYL (8-(sec-butyl)-5,6,7,8-tetrahydroquinoline); BOISAMBRENE FORTE™ ((ethoxymethoxy)cyclododecane); BOISIRIS™ ((1S,2R,5R)-2-ethoxy-2,6,6-trimethyl-9-methylenebicyclo[3.3.1]nonane); BORNYL ACETATE ((2S,4S)-1,7,7-trimethylbicyclo[2.2.1]heptan-2-yl acetate); BUTYL BUTYRO LACTATE (1-butoxy-1-oxopropan-2-yl butyrate); BUTYL CYCLOHEXYL ACETATE PARA (4-(tert-butyl)cyclohexyl acetate); CARYOPHYLLENE ((Z)-4,11,11-trimethyl-8-methylenebicyclo[7.2.0]undec-4-ene); CASHMERAN™ (1,1,2,3,3-pentamethyl-2,3,6,7-tetrahydro-1H-inden-4(5H)-one); CASSYRANE™ (5-tert-butyl-2-methyl-5-propyl-2H-furan); CITRAL ((E)-3,7-dimethylocta-2,6-dienal); CITRAL LEMAROME™ N ((E)-3,7-dimethylocta-2,6-dienal); CITRATHAL™ R ((Z)-1,1-diethoxy-3,7-dimethylocta-2,6-diene); CITRONELLAL (3,7-dimethyloct-6-enal); CITRONELLOL (3,7-dimethyloct-6-en-1-ol); CITRONELLYL ACETATE (3,7-dimethyloct-6-en-1-yl acetate); CITRONELLYL FORMATE (3,7-dimethyloct-6-en-1-yl formate); CITRONELLYL NITRILE (3,7-dimethyloct-6-enenitrile); CITRONELLYL PROPIONATE (3,7-dimethyloct-6-en-1-yl propionate); CLONAL (dodecanenitrile); CORANOL (4-cyclohexyl-2-methylbutan-2-ol); COSMONE™ ((Z)-3-methylcyclotetradec-5-enone); CYCLAMEN ALDEHYDE (3-(4-isopropylphenyl)-2-methylpropanal); CYCLOGALBANATE (allyl 2-(cyclohexyloxy)acetate); CYCLOHEXYL SALICYLATE (cyclohexyl 2-hydroxybenzoate); CYCLOMYRAL (8,8-dimethyl-1,2,3,4,5,6,7,8-octahydronaphthalene-2-carbaldehyde); DAMASCENONE ((E)-1-(2,6,6-trimethylcyclohexa-1,3-dien-1-yl)but-2-en-1-one); DAMASCONE ALPHA ((E)-1-(2,6,6-trimethylcyclohex-2-en-1-yl)but-2-en-1-one); DAMASCONE DELTA ((E)-1-(2,6,6-trimethylcyclohex-3-en-1-yl)but-2-en-1-one); DECENAL-4-TRANS ((E)-dec-4-enal); DELPHONE (2-pentylcyclopentanone); DIHYDRO ANETHOLE (propanedioic acid 1-(1-(3,3-dimethylcyclohexyl)ethyl) 3-ethyl ester); DIHYDRO JASMONE (3-methyl-2-pentylcyclopent-2-enone); DIMETHYL BENZYL CARBINOL (2-methyl-1-phenylpropan-2-ol); DIMETHYL BENZYL CARBINYL ACETATE (2-methyl-1-phenylpropan-2-yl acetate); DIMETHYL BENZYL CARBINYL BUTYRATE (2-methyl-1-phenylpropan-2-yl butyrate); DIMETHYL OCTENONE (4,7-dimethyloct-6-en-3-one); DIMETOL (2,6-dimethylheptan-2-ol); DIPENTENE (1-methyl-4-(prop-1-en-2-yl)cyclohex-1-ene); DUPICAL™ ((E)-4-((3aS,7aS)-hexahydro-1H-4,7-methanoinden-5(6H)-ylidene)butanal); EBANOL™ ((E)-3-methyl-5-(2,2,3-trimethylcyclopent-3-en-1-yl)pent-4-en-2-ol); ETHYL CAPROATE (ethyl hexanoate); ETHYL CAPRYLATE (ethyl octanoate); ETHYL LINALOOL ((E)-3,7-dimethylnona-1,6-dien-3-ol); ETHYL LINALYL ACETATE ((Z)-3,7-dimethylnona-1,6-dien-3-yl acetate); ETHYL OENANTHATE (ethyl heptanoate); ETHYL SAFRANATE (ethyl 2,6,6-trimethylcyclohexa-1,3-diene-1-carboxylate); EUCALYPTOL ((1s,4s)-1,3,3-trimethyl-2-oxabicyclo[2.2.2]octane); FENCHYL ACETATE ((2S)-1,3,3-trimethylbicyclo[2.2.1]heptan-2-yl acetate); FENCHYL ALCOHOL ((1S,2R,4R)-1,3,3-trimethylbicyclo[2.2.1]heptan-2-ol); FIXOLIDE™ (1-(3,5,5,6,8,8-hexamethyl-5,6,7,8-tetrahydronaphthalen-2-yl)ethanone); FLORALOZONE™ (3-(4-ethylphenyl)-2,2-dimethylpropanal); FLORHYDRAL (3-(3-isopropylphenyl)butanal); FLOROCYCLENE™ ((3aR,6S,7aS)-3a,4,5,6,7,7a-hexahydro-1H-4,7-methanoinden-6-yl propionate); FLOROPAL™ (2,4,6-trimethyl-4-phenyl-1,3-dioxane); FRESKOMENTHE™ (2-(sec-butyl)cyclohexanone); FRUITATE ((3aS,4S,7R,7aS)-ethyl octahydro-1H-4,7-methanoindene-3a-carboxylate); FRUTONILE (2-methyldecanenitrile); GALBANONE™ PURE (1-(3,3-dimethylcyclohex-1-en-1-yl)pent-4-en-1-one); GARDOCYCLENE™ ((3aR,6S,7aS)-3a,4,5,6,7,7a-hexahydro-1H-4,7-methanoinden-6-yl isobutyrate); GERANIOL ((E)-3,7-dimethylocta-2,6-dien-1-ol); GERANYL ACETATE SYNTHETIC ((E)-3,7-dimethylocta-2,6-dien-1-yl acetate); GERANYL ISOBUTYRATE ((E)-3,7-dimethylocta-2,6-dien-1-yl isobutyrate); GIVESCONE™ (ethyl 2-ethyl-6,6-dimethylcyclohex-2-enecarboxylate); HABANOLIDE™ ((E)-oxacyclohexadec-12-en-2-one); HEDIONE™ (methyl 3-oxo-2-pentylcyclopentaneacetate); HERBANATE™ ((2S)-ethyl 3-isopropylbicyclo[2.2.1]hept-5-ene-2-carboxylate); HEXENYL-3-CIS BUTYRATE ((Z)-hex-3-en-1-yl butyrate); HEXYL CINNAMIC ALDEHYDE ((E)-2-benzylideneoctanal); HEXYL ISOBUTYRATE (hexyl isobutyrate); HEXYL SALICYLATE (hexyl 2-hydroxybenzoate); INDOFLOR™ (4,4a,5,9b-tetrahydroindeno[1,2-d][1,3]dioxine); IONONE BETA ((E)-4-(2,6,6-trimethylcyclohex-1-en-1-yl)but-3-en-2-one); IRISONE ALPHA ((E)-4-(2,6,6-trimethylcyclohex-2-en-1-yl)but-3-en-2-one); IRONE ALPHA ((E)-4-(2,5,6,6-tetramethylcyclohex-2-en-1-yl)but-3-en-2-one); ISO E SUPER™ (1-(2,3,8,8-tetramethyl-1,2,3,4,5,6,7,8-octahydronaphthalen-2-yl)ethanone); ISOCYCLOCITRAL (2,4,6-trimethylcyclohex-3-enecarbaldehyde); ISONONYL ACETATE (3,5,5-trimethylhexyl acetate); ISOPROPYL METHYL-2-BUTYRATE (isopropyl 2-methyl butanoate); ISORALDEINE™ 70 ((E)-3-methyl-4-(2,6,6-trimethylcyclohex-2-en-1-yl)but-3-en-2-one); JASMACYCLENE™ ((3a R,6S,7aS)-3a,4,5,6,7,7a-hexahydro-1H-4,7-methanoinden-6-yl acetate); JASMONE CIS ((Z)-3-methyl-2-(pent-2-en-1-yl)cyclopent-2-enone); KARANAL™ (5-(sec-butyl)-2-(2,4-dimethylcyclohex-3-en-1-yl)-5-methyl-1,3-dioxane); KOAVONE ((Z)-3,4,5,6,6-pentamethylhept-3-en-2-one); LEAF ACETAL ((Z)-1-(1-ethoxyethoxy)hex-3-ene); LEMONILE™ ((2E,6Z)-3,7-dimethylnona-2,6-dienenitrile); LIFFAROME™ GIV ((Z)-hex-3-en-1-yl methyl carbonate); LILIAL™ (3-(4-(tert-butyl)phenyl)-2-methylpropanal); LINALOOL (3,7-dimethylocta-1,6-dien-3-ol); LINALYL ACETATE (3,7-dimethylocta-1,6-dien-3-yl acetate); MAHONIAL™ ((4E)-9-hydroxy-5,9-dimethyl-4-decenal); MALTYL ISOBUTYRATE (2-methyl-4-oxo-4H-pyran-3-yl isobutyrate); MANZANATE (ethyl 2-methylpentanoate); MELONAL™ (2,6-dimethylhept-5-enal); MENTHOL (2-isopropyl-5-methylcyclohexanol); MENTHONE (2-isopropyl-5-methylcyclohexanone); METHYL CEDRYL KETONE (1-((1S,8aS)-1,4,4,6-tetramethyl-2,3,3a,4,5,8-hexahydro-1H-5,8a-methanoazulen-7-yl)ethanone); METHYL NONYL KETONE EXTRA (undecan-2-one); METHYL OCTYNE CARBONATE (methyl non-2-ynoate); METHYL PAMPLEMOUSSE (6,6-dimethoxy-2,5,5-trimethylhex-2-ene); MYRALDENE (4-(4-methylpent-3-en-1-yl)cyclohex-3-enecarbaldehyde); NECTARYL (2-(2-(4-methylcyclohex-3-en-1-yl)propyl)cyclopentanone); NEOBERGAMATE™ FORTE (2-methyl-6-methyleneoct-7-en-2-yl acetate); NEOFOLIONE™ ((E)-methyl non-2-enoate); NEROLIDYLE™ ((Z)-3,7,11-trimethyldodeca-1,6,10-trien-3-yl acetate); NERYL ACETATE HC ((Z)-3,7-dimethylocta-2,6-dien-1-yl acetate); NONADYL (6,8-dimethylnonan-2-ol); NONENAL-6-CIS ((Z)-non-6-enal); NYMPHEAL™ (3-(4-isobutyl-2-methylphenyl)propanal); ORIVONE™ (4-(tert-pentyl)cyclohexanone); PARADISAMIDE™ (2-ethyl-N-methyl-N-(m-tolyl)butanamide); PELARGENE (2-methyl-4-methylene-6-phenyltetrahydro-2H-pyran); PEONILE™ (2-cyclohexylidene-2-phenylacetonitrile); PETALIA™ (2-cyclohexylidene-2-(o-tolyl)acetonitrile); PIVAROSE™ (2,2-dimethyl-2-pheylethyl propanoate); PRECYCLEMONE™ B (1-methyl-4-(4-methylpent-3-en-1-yl)cyclohex-3-enecarbaldehyde); PYRALONE™ (6-(sec-butyl)quinoline); RADJANOL™ SUPER ((E)-2-ethyl-4-(2,2,3-trimethylcyclopent-3-en-1-yl)but-2-en-1-ol); RASPBERRY KETONE (N112) (4-(4-hydroxyphenyl)butan-2-one); RHUBAFURANE™ (2,2,5-trimethyl-5-pentylcyclopentanone); ROSACETOL (2,2,2-trichloro-1-phenylethyl acetate); ROSALVA (dec-9-en-1-ol); ROSYFOLIA ((1-methyl-2-(5-methylhex-4-en-2-yl)cyclopropyl)-methanol); ROSYRANE™ SUPER (4-methylene-2-phenyltetrahydro-2H-pyran); SERENOLIDE (2-(1-(3,3-dimethylcyclohexyl)ethoxy)-2-methylpropyl cyclopropanecarboxylate); SILVIAL™ (3-(4-isobutylphenyl)-2-methylpropanal); SPIROGALBANONE™ (1-(spiro[4.5]dec-6-en-7-yl)pent-4-en-1-one); STEMONE™ ((E)-5-methylheptan-3-one oxime); SUPER MUGUET™ ((E)-6-ethyl-3-methyloct-6-en-1-ol); SYLKOLIDE™ ((E)-2-((3,5-dimethylhex-3-en-2-yl)oxy)-2-methylpropyl cyclopropanecarboxylate); TERPINENE GAMMA (1-methyl-4-propan-2-ylcyclohexa-1,4-diene); TERPINOLENE (1-methyl-4-(propan-2-ylidene)cyclohex-1-ene); TERPINYL ACETATE (2-(4-methylcyclohex-3-en-1-yl)propan-2-yl acetate); TETRAHYDRO LINALOOL (3,7-dimethyloctan-3-ol); TETRAHYDRO MYRCENOL (2,6-dimethyloctan-2-ol); THIBETOLIDE (oxacyclohexadecan-2-one); TRIDECENE-2-NITRILE ((E)-tridec-2-enenitrile); UNDECAVERTOL ((E)-4-methyldec-3-en-5-ol); VELOUTONE™ (2,2,5-trimethyl-5-pentylcyclopentanone); VIRIDINE™ ((2,2-dimethoxyethyl)benzene); ZINARINE™ (2-(2,4-dimethylcyclohexyl)pyridine); and mixtures thereof.

A comprehensive list of perfume ingredients that may be encapsulated in accordance with the present invention can be found in the perfumery literature, for example “Perfume & Flavor Chemicals”, S. Arctander, Allured Publishing, 1994.

In particular embodiments of the present invention, the core may also comprise a cosmetic ingredients.

Preferably, the cosmetic ingredients have a calculated octanol/water partition coefficient (Clog P) of 1.5 or more, more preferably 3 or more. Alternatively preferred, the Clog P of the cosmetic ingredient is from 2 to 7.

Particularly useful cosmetic ingredients may be selected from the group consisting of emollients, smoothening actives, hydrating actives, soothing and relaxing actives, decorative actives, anti-aging actives, draining actives, remodelling actives, skin levelling actives, preservatives, anti-oxidant actives, antibacterial or bacteriostatic actives, cleansing actives, lubricating actives, structuring actives, hair conditioning actives, whitening actives, texturing actives, softening actives, anti-dandruff actives and exfoliating actives.

Particularly useful cosmetic ingredients include, but are not limited to, hydrophobic polymers, such as alkyldimethylsiloxanes, polymethylsilsesquioxanes, polyethylene, polyisobutylene, styrene-ethylene-styrene and styrene-butylene-styrene block copolymers, mineral oils, such as hydrogenated isoparaffins, silicone oils, vegetable oils, such as argan oil, jojoba oil, aloe vera oil, fatty acids and fatty alcohols and their esters, glycolipides, phospholipides, sphingolipides, such as ceramides, sterols and steroids, terpenes, sesquiterpenes, triterpenes and their derivatives, essential oils, such as arnica oil, artemisia oil, bark tree oil, birch leaf oil, calendula oil, cinnamon oil, echinacea oil, eucalyptus oil, ginseng oil, jujube oil, helianthus oil, jasmine oil, lavender oil, lotus seed oil, perilla oil, rosmary oil, sandal wood oil, tea tree oil, thyme oil, valerian oil, wormwood oil, ylang ylang oil, yucca oil.

The resultant encapsulated composition, presented in the form of a slurry of microcapsules suspended in an aqueous suspending medium may be incorporated as such in a consumer product base. If desired, however, the slurry may be dried to present the encapsulated composition in dry powder form. Drying of a microcapsule slurry is conventional, and may be carried out according techniques known in the art, such as spray-drying, evaporation, lyophilization or use of a desiccant. Typically, as is conventional in the art, dried microcapsules will be dispersed or suspended in a suitable powder, such as powdered silica, which can act as a bulking agent or flow aid. Such suitable powder may be added to the encapsulated composition before, during or after the drying step.

A third aspect of the present invention provides an encapsulated composition obtainable by any of the methods described herein above.

A fourth aspect of the present invention relates to a use of an encapsulated composition as described herein above to enhance the performance of a perfume and/or cosmetic ingredient in a consumer good.

A fifth aspect of the present invention refers to a consumer good, in particular a consumer good suitable for use in rinse-off applications, comprising an encapsulated composition as described herein above. The consumer good is preferably selected from the group consisting of fabric care detergents and conditioners, hair care conditioners, shampoos, heavy duty liquid detergents, hard surface cleaners, detergent powders, soaps, shower gels and skin care products.

Encapsulated compositions according to the present invention are particularly useful when employed as perfume delivery vehicles in consumer goods that require, for delivering optimal perfumery benefits, that the microcapsules adhere well to a substrate on which they are applied. Such consumer goods include hair shampoos and conditioners, as well as textile-treatment products, such as laundry detergents and conditioners.

A sixth aspect of the present invention relates to a polymeric stabilizer, which is formed by combination of a polymeric surfactant with a bipodal aminosilane and optionally a further aminosilane, in particular for use as first shell in core-shell microcapsule formation. This first shell stabilizes the microcapsule core-water interface and is sufficiently impervious to perfume ingredients to be used as sole encapsulating shell. It may also be encapsulated or partially encapsulated by a second shell that may provide additional stability and/or additional functionalities. Furthermore, a coating may be applied on the first or second shell to also provide additional stability and/or additional functionalities.

A seventh aspect of the present invention refers to a use of a polymeric stabilizer as described herein above in the encapsulation of perfume and/or cosmetic ingredients. The polymeric stabilizer stabilizes the oil/water interfaces and, thereby, provides a template for the preparation of encapsulated perfume and/or cosmetic compositions.

The present disclosure also relates to a method for enhancing the performance of a perfume and/or cosmetic ingredient in a consumer product by adding an encapsulated composition according to the present invention.

Furthermore, the present disclosure refers to a method of encapsulating a perfume and/or cosmetic ingredient, wherein the polymeric stabilizer as described herein above stabilizes and encapsulates the oil droplets of the oil in water emulsion and wherein the oil phase comprises the at least one perfume and/or cosmetic ingredient.

There now follows a series of examples that serve to further illustrate the present invention.

### EXAMPLE 1

Formation of microcapsules having first shell comprising a polymeric stabilizer according to the present invention:

In EXAMPLES 1.1 to 1.6, a series of core-shell microcapsules have been obtained, wherein the levels of polymeric surfactant (of ZeMac E400, ex Vertellus), bipodal aminosilane (bis(3-(triethoxysilyl)propyl)amine) and further aminosilane (((trimethoxysilyl)propyl)anilin) have been varied according to Table 1.

The microcapsules have been obtained by performing the steps of:
a. Preparing a core composition comprising a well-defined amount (see Table 1) of bipodal aminosilane (bis(3-(triethoxysilyl)propyl)amine) and a well-defined amount (see Table 1) of further aminosilane (((trimethoxysilyl)propyl)anilin) by admixing both aminosilanes with 40 g of fragrance composition;
b. Emulsifying the core composition obtained in step a. in a mixture of a well-defined amount (see Table 1) of ZeMac E400 in 39 g of water by using a 300 ml reactor and a cross-beam stirrer with pitched beam operating at a stirring speed of 600 rpm at a temperature of 25±2° C.;
c. Adjusting the pH of the continuous phase of the emulsion to 4.4±0.5 with a 20% NH₃ solution in water and maintaining the system at a temperature of 25±2° C. for 3.5 h while maintaining stirring as in step b.;
d. Increasing the temperature to 80° C. for 1 h while maintaining stirring as in steps b. and c. to complete the formation of core-shell capsules;
e. Letting the slurry of core-shell capsules obtained in step d. cool to room temperature.

The solid content of each of the slurries was measured by using a thermo-balance operating at 120° C. The solid content, expressed as weight percentage of the initial slurry deposited on the balance was taken at the point where the drying-induced rate of weight change had dropped below 0.1%/min. The ratio of the measured solid content to the theoretical solid content calculated based on the weight of perfume and encapsulating materials involved is taken as a measurement of encapsulation yield, expressed in wt.-%.

TABLE 1
Formulation details for EXAMPLES 1.1 through 1.6

| | Dipodal | Further | ZeMac | Encapsulation |
|---|---|---|---|---|
| | aminosilane | aminosilane | E400 | yield |
| | [wt.-%] | [wt.-%] | [wt.-%] | [wt.-%] |
| EXAMPLE 1.1 | 0.2 | — | 1.5 | 40 |
| EXAMPLE 1.2 | 0.5 | — | 1.5 | 90 |
| EXAMPLE 1.3 | 0.5 | — | 0.75 | <20 |
| EXAMPLE 1.4 | 1.0 | — | 1.5 | 100 |
| EXAMPLE 1.5 | — | 1.0 | 1.5 | <20 |
| EXAMPLE 1.6 | 0.5 | 0.5 | 1.5 | 100 |

As apparent from encapsulation yield, the presence of the dipodal aminosilane is a prerequisite for obtaining core-shell microcapsules having a high encapsulation yield. If the dipodal aminosilane is used alone with ZeMac E400, then optimal dipodal aminosilane to ZeMac E400 weight ratio range is from 0.3 to 0.7. If the dipodal aminosilane is used along with a further aminosilane and ZeMac E400, then the optimal dipodal aminosilane to ZeMac E400 weight ratio range is from 0.3 to 0.5 and the further aminosilane to polymer surfactant weight ratio in the emulsion is set within a range of from 0.3 to 0.5.

### EXAMPLE 2

In EXAMPLE 2, microcapsules according to the prior art were prepared using the method described in US 2014/0331414 A1:

462 g of water, 15 g of formic acid and 250 g of a 10% polyvinylpyrrolidone solution were introduced into a 1 L reactor under stirring. The stirring speed was set to 600 rpm and 200 g of perfume was added, followed by 44.6 g of methyl triethoxysilane, 16.4 g of tetraethoxysilane, 11.4 g of dimethyldiethoxysilane and 2.5 g of aminopropyltriethoxysilane at room temperature. After 2 h, the pH was slowly increased to 6 with a 20% sodium hydroxide solution and the temperature increased to 80° C. After 4 h at 80° C., the microcapsule slurry was slowly cooled to 25° C. A slurry of microcapsules of solid contents of 26% was obtained. The microcapsules had an average particle size of 17 micrometers.

### EXAMPLE 3

In EXAMPLE 3, microcapsules having a first shell comprising a polymeric stabilizer according to the present invention and a second shell comprising an aminoplast resin were prepared by performing the steps of:
a. Preparing microcapsules comprising the polymeric stabilizer as described in EXAMPLE 1.6;
b. Adding 0.75 g of urea and 1.15 g of Luracoll SD (methylolated melamine pre-condensates ex. BASF), under continuous and stirring for 30 minutes at 35° C.;
c. Increasing the temperature to 60° C. for 1 h and then to 80° C. for 1 h, while maintaining under stirring to obtain a slurry of microcapsules having a second shell of aminoplast resin surrounding the first shell comprising the polymeric stabilizer;
d. Cooling down the slurry to room temperature.

### EXAMPLE 4

In EXAMPLE 4, microcapsules having a first shell comprising a polymeric stabilizer according to the present invention and a second polyurea-based shell were prepared by performing the steps of:
a. Preparing microcapsules comprising the polymeric stabilizer as described in EXAMPLE 1.6;
b. Adding 2 g of hydrodispersible isocyanate based on hexamethylene diisocyanate (Bayhydur® XP2547, Covestro) and 22 g of diisocyanate 4,4-dicyclohexylmethanediyle (Desmodur® W1, Covestro) to the emulsion, while maintaining the system stirring as in step b. and c. of Example 1 at a temperature of 35±2° C. for 30 minutes;
c. Adding 8 g of polyethyleneimine solution (Lupasol® G100 at 35% by weight in deionized water, BASF) in one step and heating reaction mixture gradually to 70° C. during 2 h;
d. Adding 12.5 g of a 40 wt.-% aqueous solution of copolymer of acrylic acid and diallyldimethylammonium chloride (Merquat 281, Lubrizol) and further heating the reaction mixture to 85° C. for 2 h;
e. Adding 410 g of ammonia solution and 3 g hydroxyethyl cellulose (Natrosol™ 250 HX, Ashland) and cooling down the mixture to room temperature.
f. Adjusting the final pH of the suspension to 4.0±0.2 with citric acid solution.

### EXAMPLE 5

### Assessment of Leakage of Microcapsules in a Model Extractive Medium:

The model extractive medium was a system consisting of an aqueous solution of ethanol at an initial concentration of 30 vol.-% co-existing with an immiscible cyclohexane phase.

In a first step, 10 ml of cyclohexane was put into a vial. Then 1.8 ml of the 30 vol.-% ethanol in water was added to the vial. After equilibration, taking into account the partition coefficient of ethanol between cyclohexane and the water of 0.03, the percentage of ethanol in the aqueous phase was 25.2±0.5 vol.-% and the percentage of ethanol referred to the whole system was 2.4±0.05 vol.-%.

In a second step, the slurry to be assessed was diluted in such a way that the perfume concentration in the diluted slurry was about 10 wt.-% and 200 microliters of this diluted slurry was added to the vial.

In a third step, the vial was submitted to a horizontal mixing on an elliptic xy-mixing equipment operating at a 250 rpm for 4 h (shaking in the z direction was avoided).

In a fourth step, the upper cyclohexane phase containing the extracted perfume was analysed spectrophotometrically by using a UV/visible light spectrometer. The perfume concentration was determined by measuring the intensity of the absorbed UV/visible light at the maximum absorbance wavelength, which has been determined previously by using a reference perfume/cyclohexane solution of known concentration. This latter reference solution was used as an external standard for the quantification of the extracted perfume. The leakage value is defined as the percentage of the encapsulated perfume that has been recovered in the hexane phase.

Representative leakage values are given in Table 2, hereunder.

### EXAMPLE 6

### Assessment of Fragrance Release Performance:

The release performance of the microcapsule slurries was measured by using a texture analyzer (TA XT PLUS, ex TA instruments). 300 microliters of undiluted slurry were deposited on the surface of filter paper in three successive applications of 100 microliters and left to dry overnight. Then, the lower surface of a mechanical sensor probe, consisting of a flat metal cylinder having a diameter of 12.5 micrometer, was applied on the deposited microcapsules with a penetration velocity of 0.01 mm/s.

As the probe penetrates the bed of microcapsules deposited on the filter paper, it experiences a back elastic force which is proportional to the elastic bending modulus of the microcapsules, which is inversely proportional to the release performance of the microcapsules. The value of the measured force at the 50% deformation of the microcapsule bed is taken as a measurement of the release performance of the microcapsules. The displacement corresponding to 50% deformation point is determined as the half way point between the displacement point where the first contact with the microcapsules occurs, which is marked by the onset of a back force and the point where the probe motion is stopped by the filter paper.

TABLE 2
Perfume leakage in water/ethanol/cyclohexane and
force at 50% deformation for selected examples

| | Leakage at 30 vol.-% EtOH | Force at 50% |
|---|---|---|
| Example | [wt.-%] | deformation [N] |
| EXAMPLE 1.4 | 50 | 1.7 |
| EXAMPLE 1.6 | 25 | 3.0 |
| EXAMPLE 2 | 100 | 2.9 |
| EXAMPLE 3 | <20 | 7.2 |
| EXAMPLE 4 | <20 | 6.9 |

It may be concluded from the value of the force at 50% deformation measured on the microcapsules of EXAMPLE 1.4 that the shells comprising the new polymeric stabilizer are solid and have a measurable elastic modulus. This result, combined with the limited leakage in model extractive medium, confirms that an encapsulating shell has been effectively formed in this example. The values for EXAMPLE 1.6 confirm that adding a further aminosilane effectively improves both mechanical stability and stability with respect to leakage. The leakage values of EXAMPLE 3 and 4 confirm the stabilizing effect of adding a second aminoplast shell. Finally, comparison with prior art microcapsules comprising conventional alkoxysilanes and aminopropyltriethoxysilane shows that the latter are much less stable with respect to leakage.

### EXAMPLE 7

### Comparison of Olfactive Performance of New and Conventional Silane-Based Microcapsules:

The olfactive performance of microcapsules of EXAMPLES 1.6, 3 and 4 according to the present invention have been compared with the olfactive performance of conventional silane-based microcapsules according to EXAMPLE 2. The samples were evaluated in a unperfumed hair care conditioner. The aforementioned microcapsule slurries were added to a hair care conditioner composition under gentle stirring with a paddle mixer, so that the level of slurry in the hair care conditioner base was 1 wt.-% referred to the total weight of the hair care conditioner base. 1.5 g of hair care conditioner was applied on 15 g swatches humidified with 12 g water. The swatches were submitted to a massage, left to stand for 1 minute and then rinsed 30 seconds under running tap water at 37° C. at a flow rate of 3.2 l/min, without touching the swatch by hand. The pre-rub olfactive evaluation was performed on the swatches after 4 h. For this evaluation, the swatches were handled carefully in order to minimize the risk of breaking the microcapsules mechanically. The post-rub olfactive evaluation was performed after drying the swatches for 24 h at room temperature. This evaluation was performed by gently rubbing one part of each swatch. The olfactive performance (intensity) was assessed by a panel of 4 experts rated on a scale of 1-5 (1=barely noticeable, 2=weak, 3=medium, 4=strong and 5=very strong). When relevant, qualitative comments on the perceived odor direction were recorded.

This evaluation was performed on fresh samples and on samples that have been stored for one month at 37° C.

TABLE 3
Olfactive performance on hair swatch of
freshly prepared and aged microcapsules

| | Pre-rub | Pre-rub | Post-rub | Post-rub |
|---|---|---|---|---|
| | intensity | intensity | intensity | intensity |
| | (fresh | (aged | (fresh | (aged |
| | sample) | sample) | sample) | sample) |
| EXAMPLE 1.6 | 1.9 | 1.6 | 4.1 | 3.8 |
| EXAMPLE 2 | 3.1 | 0.4 | 3.3 | 0.5 |
| EXAMPLE 3 | 3.6 | 3.1 | 4.6 | 4.0 |
| EXAMPLE 4 | 3.4 | 2.9 | 4.4 | 3.9 |

The results show that microcapsules according to the present invention provide significant enhancement of the perfume performance compared to conventional aminoplast silane-based microcapsules.

1. An encapsulated composition comprising at least one core-shell microcapsule, wherein the at least one core-shell microcapsule comprises a core comprising at least one perfume and/or cosmetic ingredient, and a shell surrounding the core, wherein the shell comprises a polymeric stabilizer that is formed by combination of a polymeric surfactant with at least one bipodal aminosilane.

2. The encapsulated composition according to claim 1, wherein the bipodal aminosilane is an aminosilane of Formula (I)
(O—R⁴)_{(3-f)}(R³)_{f}Si—R²—X—R²—Si(O—R⁴)_{(3-f)}(R³)_{f} Formula (I)
wherein X stands for —NR¹—, —NR¹—CH₂—NR¹—, —NR¹—CH₂—CH₂—NR¹—, —NR¹—CO—NR¹— or 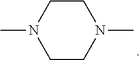
R¹ each independently stands for H, CH₃ or C₂H₅;
R² each independently stands for a linear or branched alkylene group with 1 to 6 carbon atoms;
R³ each independently stands for a linear or branched alkyl group with 1 to 4 carbon atoms;
R⁴ each independently stands for H or for a linear or branched alkyl group with 1 to 4 carbon atoms;
f stands for 0, 1 or 2.

3. The encapsulated composition according to claim 2, wherein the bipodal aminosilane is selected from the group consisting of bis(3-(triethoxysilyl)propyl)amine, N,N′-bis(3-(trimethoxysilyl)propyl)urea, bis(3-(methyldiethoxysilyl)propyl)amine, N,N′-bis(3-(trimethoxysilyl)propyl)ethane-1,2-diamine, bis(3-(methyldimethoxysilyl)propyl)-N-methylamine and 1,4-bis(3-(triethoxysilyl)propyl)piperazine.

4. The encapsulated composition according to claim 1, wherein the bipodal aminosilane is a secondary aminosilane.

5. The encapsulated composition according to claim 1, wherein the polymeric stabilizer is formed by combination of the polymeric surfactant with the at least one bipodal aminosilane and a further aminosilane, preferably an aromatic aminosilane, even more preferably selected from the group consisting of compounds having Formula II 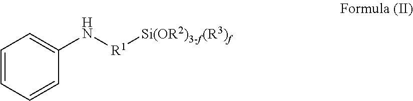
wherein R¹ stands for a linear or branched alkylene group with 1 to 6 carbon atoms;
R² each independently stands for a linear or branched alkyl group with 1 to 4 carbon atoms;
R³ each independently stands for H or for a linear or branched alkyl group with 1 to 4 carbon atoms;
f stands for 0, 1, or 2.

6. The encapsulated composition according to claim 5, wherein the aromatic aminosilane is selected from the group consisting of N-(3-(trimethoxysilyl)propyl)aniline and N-((trimethoxysilyl)methy)aniline.

7. The encapsulated composition according to claim 1, wherein the polymeric surfactant is a co-polymer of maleic anhydride and ethylene and/or vinyl methyl ether.

8. The encapsulated composition according to claim 7, wherein the co-polymer of maleic anhydride and ethylene and/or vinyl methyl ether is alternate.

9. The encapsulated composition according to claim 7, wherein the co-polymer of maleic anhydride and ethylene and/or vinyl methyl ether is fully or partially hydrolyzed.

10. The encapsulated composition according to claim 1, wherein the polymeric stabilizer is formed by combination of bis(3-(triethoxysilyl)propyl)amine with (3-(trimethoxysilyl)propyl)aniline and fully or partially hydrolyzed poly(ethylene-maleic anhydride) and/or poly(vinylmethylether-maleic anhydride).

11. The encapsulated composition according to claim 1, wherein the bipodal aminosilane to polymeric surfactant weight ratio is from 0.02 to 1.

12. The encapsulated composition according to claim 5, wherein the further aminosilane to polymeric surfactant weight ratio is from 0.02 to 1.

13. The encapsulated composition according to claim 1, wherein the shell additionally comprises at least one shell-forming material obtainable by:
reacting an alkylolated polyfunctional amine or reacting a polyfunctional amine an aldehyde;
reacting a polyisocyanate and a polyfunctional amine;
reacting a polyfunctional amine and a polyfunctional (meth)acrylate; and/or
reacting unsaturated monomers selected from the group consisting of styrene, divinylbenzene, alkyl (meth)acrilyates, polyfunctional (meth)acrylates, and vinyl monomes.

14. A method for preparing an encapsulated composition, in particular an encapsulated composition according to claim 1, the method comprising the steps of:
a. Dissolving a polymeric surfactant in an aqueous phase;
b. Dissolving at least one bipodal aminosilane in an oil phase comprising at least one perfume and/or cosmetic ingredient;
c. Emulsifying the oil phase into the aqueous phase to form an oil-in-water emulsion;
d. Causing the bipodal aminosilane and the polymeric surfactant to form a first shell of polymeric stabilizer encapsulating the dispersed oil droplets, thereby forming a slurry of microcapsules; and optionally:
e. Providing additional shell-forming materials and causing them to react in order to form an additional shell encapsulating the microcapsules formed in step d.

15. (canceled)

16. (canceled)

17. A consumer good comprising an encapsulated composition according to claim 1, wherein the consumer good is preferably selected from the group consisting of fabric care detergents and conditioners, hair care conditioners, shampoos, heavy duty liquid detergents, hard surface cleaners, detergent powders, soaps, shower gels and skin care products.

18. A polymeric stabilizer which is formed by combination of a polymeric surfactant with a bipodal aminosilane.

19. (canceled)

20. The encapsulated composition according to claim 11, wherein the bipodal aminosilane to polymeric surfactant weight ratio is from 0.2 to 0.9.

21. The encapsulated composition according to claim 12, wherein the further aminosilane to polymeric surfactant weight ratio is from 0.1 to 0.7.
